(19) United States
(12) Patent Application Publication
(10) Pub. No.: US 2022/0000855 A1
(43) Pub. Date: Jan. 6, 2022

(54) AN AURORA A KINASE INHIBITOR FOR USE IN THE TREATMENT OF NEUROBLASTOMA

(71) Applicant: Eli Lilly and Company, Indianapolis, IN (US)

(72) Inventors: Michele Suzanne DOWLESS, Pendleton, IN (US); Xueqian GONG, Carmel, IN (US); Louis Frank STANCATO, Fishers, IN (US)

(21) Appl. No.: 17/295,721

(22) PCT Filed: Nov. 22, 2019
(86) PCT No.: PCT/US2019/062718

§ 371 Date: May 20, 2021

Related U.S. Application Data
(60) Provisional application No. 62/773,367, filed on Nov. 30, 2018.

(51) Int. Cl. A61K 31/4545; A61P 35/00

(52) U.S. Cl. CPC A61K 31/4545; A61P 35/00

(57) ABSTRACT

The present invention provides an inhibitor of Aurora A kinase, Formula (I) illustrated below, or pharmaceutically acceptable salt thereof, for use in treating neuroblastoma.

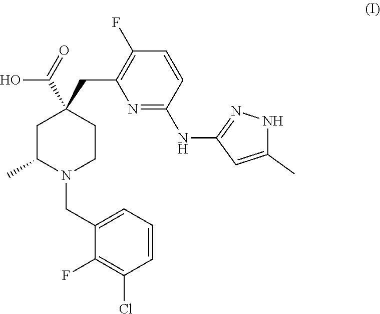

The present invention relates to the use of an Aurora A kinase inhibitor, and salts thereof, for the treatment of neuroblastoma.

Neuroblastoma is one of the most common solid tumors in children, and more than 650 neuroblastoma cases are diagnosed each year in North America. Neuroblastoma can be subdivided into two defined patient subsets, referred to generally as low risk and high risk. Low risk neuroblastoma is usually found in children younger than 18 months of age with limited disease burden resulting in a favorable prognosis. However, high-risk neuroblastoma generally occurs in children older than 18 months, frequently metastatic in bone tissue, resulting in poor prognosis. Although advances in multimodal treatment strategies have led to improved outcomes for neuroblastoma patients, survival rates for the high-risk category patients remain poor with less than 50% survival five years after diagnosis.

High-risk neuroblastoma is associated with the MYCN gene which encodes the N-myc proto-oncogene protein (N-MYC). Although incompletely understood, N-MYC and Aurora A Kinase appear to interact, and Aurora A kinase expression and amplification are thought to stabilize N-MYC and/or slow its degradation, which in turn would cause an increase in N-MYC levels. Michaelis, M, et al., “Aurora Kinases as Targets in Drug-Resistant Neuroblastoma Cells”, PLOS One, 2014, 9(9) e108758.

Aurora A kinase inhibitors are known in the art (see, for example, PCT Patent Application Publication, WO2016077191, which discloses the compound of Formula I (see below). Use of certain Aurora kinase inhibitors, including an Aurora A selective inhibitor, alisertib, and a pan Aurora inhibitor, tozasertib, have been associated with unacceptably high levels of neutropenia and other toxic effects.

A need exists for novel approaches and medications to treat neuroblastoma, in particular, high-risk neuroblastoma. In addition, there is a need to provide methods of inhibiting Aurora kinases, in particular Aurora A kinase, and decreasing the expression and/or activity of N-MYC. The present invention addresses these needs and provides a method of treating neuroblastoma.

In one form, the present invention provides a method for treating neuroblastoma in a patient in need of treatment. Preferably the present invention provides a method for treating high neuroblastoma in a patient in need of treatment. The method comprises administering to the patient an effective amount of a compound which is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, illustrated below as Formula I, or a pharmaceutically acceptable salt of the compound of Formula I. In one embodiment, the compound of Formula I is provided as a free acid. In another embodiment, the compound of Formula I is provided as a base addition salt. In one preferred embodiment, the compound of Formula I is provided as a 2-methylpropan-2-ammonium salt (also known as an erbumine salt or a tert-butylamine salt) that is ((2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methyl-2-propanamine (1:1)). In another embodiment, the compound of Formula I is provided as an ammonium salt ((2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt).

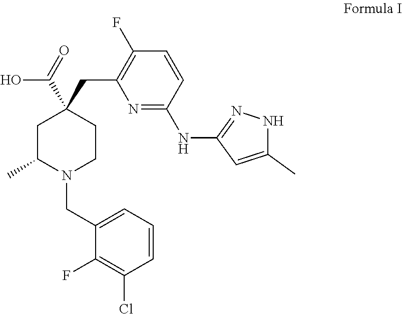

In another form, the present invention provides a pharmaceutical composition comprising the compound of Formula I, or a pharmaceutically acceptable salt thereof, and one or more of a pharmaceutically acceptable: carrier, diluent, or excipient for use in treating neuroblastoma, preferably for treating high risk neuroblastoma. In one embodiment, the composition comprises a compound of Formula I, which is free acid. In another embodiment, the composition comprises a compound of Formula I as a base addition salt, preferably, a 2-methylpropan-2-ammonium salt or an ammonium salt, more preferable a methylpropan-2-ammonium salt.

The present invention provides the compound of Formula I, or a pharmaceutically acceptable salt thereof, for use in the treatment of neuroblastoma. The present invention also provides for the use of the compound of Formula I, or a pharmaceutically acceptable salt thereof, for the manufacture of a medicament for the treatment of neuroblastoma. In one embodiment, the compound is provided as a free acid. In another embodiment, the compound of Formula I is provided as a base addition salt. In one preferred embodiment, the compound of Formula I is provided as a 2-methylpropan-2-ammonium salt. In still yet another embodiment, the compound of Formula I is provided as an ammonium salt.

The compound of Formula I, or pharmaceutically acceptable salt thereof, can be used in combination with the standard-of-care treatment for patients in need of treatment for neuroblastoma. The standard-of-care treatment can include one or more of the following: surgery or excision of all or a portion of the tumor, radiation therapy, stem cell transplant, administering a chemotherapeutic agents, differentiation agent, and immunotherapy.

Examples of additional chemotherapeutic agents that can be combined or administered with the compound of Formula I, or a pharmaceutically acceptable salt thereof include: alkylators (cyclophosphamide, temozolomide, and melphalan hydrochloride), platinum agents (carboplatin, cisplatin, and oxaliplatin), anthracyclines (doxorubicin hydrochloride), topoisomerase I inhibitors (irinotecan and topotecan), and vinca alkaloids (vincristine sulfate). Differentiation agents include isotretinoin (13-cis-retinoic acid), and immunotherapeutic agents include monoclonal antibodies such GD2 monoclonal antibodies (dinutuximab). The compound of Formula I, or a pharmaceutically acceptable salt thereof, and one or more additional chemotherapeutic agents, differentiation agents and/or immunotherapeutic agents can be administered simultaneously, separately, or sequentially to treat neuroblastoma.

The term “pharmaceutically acceptable salt” as used herein, refers to salts of the compound of Formula I. Examples of pharmaceutically acceptable salts and methods for their preparation can be found in, Stahl. P, et al., “Handbook of Pharmaceutical Salts: Properties, Selection and Use”, 2nd Revised Edition, Wiley-VCH, (2011) and Berge, S. M., et al., “Pharmaceutical Salts”, Journal of Pharmaceutical Sciences, 1977, 66(1), 1-19; Gould, P. L., “Salt selection for basic drugs”, International Journal of Pharmaceutics, 1986, 33: 201-217; and Bastin, R. J., et al. “Salt Selection and Optimization Procedures for Pharmaceutical New Chemical Entities”, Organic Process Research and Development, 2000, 4(5) 427-435.

The compound of Formula I, or a pharmaceutically acceptable salt thereof, can be formulated for administration as part of a pharmaceutical composition. Preferred pharmaceutical compositions can be formulated as a tablet or capsule for oral administration, a solution for oral administration or an injectable solution. The tablet, capsule, or solution can include the compound of Formula I, or a pharmaceutically acceptable salt thereof, in an amount effective for treating neuroblastoma in a patient in need of treatment. More preferably, such compositions are for oral administration. As such, pharmaceutical compositions comprising the compound of Formula I, or a pharmaceutically acceptable salt thereof, can be in combination with one or more pharmaceutically acceptable additives. The term “pharmaceutically acceptable additive(s)” as used herein for the pharmaceutical compositions, refers to one or more of: carriers, diluents, and excipients that are compatible with the other additives of the composition or formulation and not deleterious to the patient. Examples of pharmaceutical compositions and processes for their preparation can be found in “Remington: The Science and Practice of Pharmacy”, Loyd, V., et al. Eds., 22^{nd} Ed., Mack Publishing Co., (2012). Non-limiting examples of pharmaceutically acceptable carriers, diluents, and excipients include the following: saline, water, starch, sugars, mannitol, and silica derivatives; binding agents such as carboxymethyl cellulose, alginates, gelatin, and polyvinyl-pyrrolidone; kaolin and bentonite; and polyethyl glycols.

“Effective amount” means the amount of the compound of Formula I, or pharmaceutically acceptable salt thereof; or pharmaceutical composition containing the compound of Formula I, or pharmaceutically acceptable salt thereof, that will elicit the biological or medical response of or desired therapeutic effect on a tissue, system, animal, mammal or human that is being sought by the researcher, veterinarian, medical doctor or other clinician. In certain embodiments, the effective amount refers to the amount of the compound of Formula I, or a pharmaceutically acceptable salt, when administered that is effective to slow, stop, or reverse the progression of neuroblastoma; or slow or stop the growth or proliferation of neuroblastoma cells in a patient.

The effective amount of the compound of Formula I, or a pharmaceutically acceptable salt thereof, actually administered that will elicit the biological or medical response of or desired therapeutic effect on a tissue, system or patient will be determined by a physician under the relevant circumstances, including the condition to be treated, the chosen route of administration, the actual compound of the present invention administered, the age, weight, and response of the individual patient, and the severity of the patient's symptoms. Dosages per day normally fall within the range of about 0.1 to about 100 mg. In some instances, dosage levels below the lower limit of this range may be more than adequate, while in other cases still larger doses may be employed. Preferred dosages fall within the range of 1 to 80 mg; more preferably between 1 and 50 mg; still more preferably between 1 and 30 mg; still yet more preferably between 1 to 25 mg. The dosages can be administered once, twice, three times or more daily. In one embodiment, the compound of the present invention can be administered at a dosage of 15 mg or 25 mg per dose administered orally twice a day (BID).

As used herein, the term “patient” refers to a human or nonhuman mammal. More particularly, the term “patient” refers to a human.

The term “treating” (or “treat” or “treatment”) refers to the process involving a slowing, interrupting, arresting, controlling, reducing, or reversing the progression or severity of a symptom, disorder, condition, or disease such as neuroblastoma

As used herein, the following terms have the meanings indicated: “ATCC” refers to American Type Culture collection; “BID” refers to twice a day dosing; “DMEM” refers to Dulbecco's Modified Eagle's Medium; “DNA” refers to deoxyribonucleic acid; “EMEM” refers to Eagles's Minimal Essential Medium; “F12” refers to Ham's F12 medium; “FBS” refers to Fetal Bovine Serum; “HBSS” refers to Hank's Balanced Salt Solution; “HSRRB” refers to Health Science Research Resources Bank; “JCRB” refers to Japanese Collection of Research Bioresources; “MEM” refers to Minimum Essential Medium; “NBL” refers to neuroblastoma; “NEAA” refers to Non-Essential Amino Acids; “PBS” refers to phosphate-buffered saline; “RPMI” refers to Roswell Park Memorial Institute; and “SCID” refers to severe combined immunodeficient mice.

The compound of Formula I and pharmaceutically acceptable salts thereof including the 2-methylpropan-2-ammonium and ammonia salts can be prepared according to the synthetic methods disclosed in U.S. Pat. No. 9,637,474.

### Biological Assays Monolayer Anti-Proliferation Assays

One measure of potency of an Aurora A inhibitor is its ability to inhibit the proliferation of cancer cells in culture due to cell cycle arrest and mitotic catastrophe. Anti-proliferative activity of Aurora A inhibitor in NBL cell lines may be indicative of clinical responsiveness to Aurora A inhibitors. The NBL tumor cell lines are recovered from frozen stocks and cultured for 1-2 passages in cell culture flasks. The NBL tumor cell lines include: CHP-212, GOTO, IMR-32, NB16, NH-6, SH-SY5Y, SK-N-AS, SK-N-DZ, SK-N-F1, SK-N-MC, SK-N-SH, and TGW detailed in Table 1.

TABLE 1

| Cell line | Vendor | Catalog # | Lot. No | Histology | Complete medium |
|---|---|---|---|---|---|
| CHP-212 | ATCC | CRL-2273 | 58063161 | neuroblastoma | DMEM:F12(1:1) + 10% |
| | | | | | FBS |
| IMR32 | ATCC | CCL-127 | 59587034 | neuroblastoma | EMEM + 10% FBS |
| SK-N-AS | ATCC | CRL-2137 | 58078525 | neuroblastoma | DMEM + 0.1 mM |
| | | | | | NEAA + 10% FBS |
| NH-6 | JCRB | JCRB0832 | 06262000 | neuroblastoma | alpha-MEM + 10% |
| | | | | with | FBS |
| | | | | opsomyoclonus, | |
| | | | | truncal ataxia | |
| SK-N-DZ | ATCC | CRL-2149 | 3903996 | brain, | DMEM + 0.1 mM |
| | | | | neuroblastoma | NEAA + 10% FBS |
| SK-N-FI | ATCC | CRL-2142 | 58078707 | brain, | DMEM + 0.1 mM |
| | | | | neuroblastoma | NEAA + 10% FBS |
| SK-N-SH | ATCC | HTB-11 | 59257297 | brain, | EMEM + 10% FBS |
| | | | | neuroepithelioma | |
| GOTO | HSRRB | JCRB0612 | | neuroblastoma | RPMI1640:MEM(1:1) + |
| | | | | | 10% FBS |
| NB16 | RIKEN | RCB0478 | | neuroblastoma | RPMI1640 + 15% |
| | | | | | FBS |
| SH-SY5Y | ATCC | CRL-2266 | | neuroblastoma | MEM:F12(1:1) + 10% |
| | | | | | FBS |
| TGW | HSRRB | JCRB0618 | | neuroblastoma | MEM + 10% FBS |
| KELLY | Sigma | 92110411 | | neuroblastoma | DMEM + 0.1 mM |
| | | | | | NEAA + 10% FBS |

Anti-proliferative activity of an Aurora A inhibitor can be measured by CellTiter Glo® assay. Prior to treatment with the compound of Formula I, cells are plated in complete growth media into white walled clear bottom microtiter plates at a predetermined optimal density for each cell line. Sixteen hours after plating, the compound of Formula I is added. Two cell doubling times after compound addition, CellTiter-Glo® reagents are prepared according to the manufacturer's protocols, and added to each well. Plates are incubated at room temperature for 10 minutes then read with a luminescence plate reader according to manufacturer's protocol for CellTiter-Glo® Luminescent Cell Viability Assay, Promega Catalog #G7571.

Anti-proliferative activity of an Aurora A inhibitor can also be measured by counting cells after treatment. For this assay, NBL cell lines SK-N-DZ, SK-N-F1, and KELLY are plated in complete growth media into black walled clear bottom microtiter plates at 5,000 cells per well. Sixteen hours after plating, the compound of Formula I is added for 72 hours. Cells are then fixed in 3.7% formaldehyde (Sigma #F-1268,) permeabilized with 0.1% Triton X-100 (Roche #92522020) in PBS for 10 minutes then DNA is stained with Hoechst 33342 (Mol. Probes #H-21492) diluted 1:5000 in PBS. Stained plates are scanned with a Celllnsight NXT® screening platform (Thermo Fischer) using the target activation bioapplication to quantitate nuclei per field, a measure of cells per well. For both assays, absolute EC₅₀ values are reported from 10-point serial dilution curves of Formula I.

As illustrated in Table 2, pediatric NBL cell lines are highly sensitive to in vitro treatment with the compound of Formula I. This indicates that the compound of Formula I can be effective to inhibit the cell growth of a variety of neuroblastoma cell lines.

TABLE 2

| | | Biological | Technical | Mean |
|---|---|---|---|---|
| Cell Line | Assay | Experimental | Experimental | (ABS† |
| Name | type | Replicates | Replicates | IC₅₀) |
| CHP-212 | CTG* | 6 | | 0.085 |
| GOTO | CTG | 4 | | 20.000 |
| IMR-32 | CTG | 4 | | 0.016 |
| NB16 | CTG | 4 | | 0.035 |
| NH-6 | CTG | 4 | | 0.031 |
| SH-SY5Y | CTG | 4 | | 0.047 |
| SK-N-AS | CTG | 4 | | 0.823 |
| SK-N-DZ | CTG | 4 | | 0.044 |
| SK-N-DZ | Imaging‡ | | 4 | 0.135 |
| SK-N-FI | CTG | 4 | | 0.098 |
| SK-N-FI | Imaging | | 4 | 0.290 |
| SK-N-MC | CTG | 4 | | 0.873 |
| SK-N-SH | CTG | 4 | | 0.078 |
| TGW | CTG | 4 | | 0.154 |
| KELLY | Imaging | | 4 | 0.396 |

*CTG refers to CellTiter-Glo ® Luminescent Cell Viability Assay performed at HDBiosciences;
†ABS means absolute;
‡Imaging = anti-proliferation assay measured by cell counts (nuclear staining)

### Single Agent Efficacy in Neuroblastoma Xenograft Tumor Models

The efficacy of the compound of Formula I, or a pharmaceutically acceptable salt thereof, can be evaluated in in vivo mouse models of neuroblastoma. The compound of Formula I as 2-methyl-2-propanamine salt (34.5 mg/kg) can be administered orally to nude or C.B-17 SCID mice bearing cell-derived xenografts (CDX) using a 28 day BID dosing schedule. Tumor volume and body weight can be measured two times per week.

The following protocol can be used to measure reductions in tumor volume in response to an active pharmaceutical ingredient. Expand human NBL cancer cells in culture, harvest cycles and inject 5×10⁶ cells in 200 μL of 1:1 solution of HBSS and Matrigel® subcutaneously into the right rear flank of female mice (20-24 g, Charles River Laboratories). The following cell line mouse strain combinations are used: SH-SY5Y (ATCC, #CRL-2226) in Athymic nude mice, KELLY (Sigma-#92110411) in C.B.-17 SCID mice, and IMR-32 (ATCC, #CCL-127) in C.B.-17 SCID mice.

Formulate the compound of Formula I as the 2-methyl-2-propanamine salt in 20% 2-hydroxypropyl-β-cyclodextrin in 25 mM phosphate buffer, pH 2 and dose orally at 34.5 mg/kg BID for 28 days. Measure body weight and tumor volume two times per week.

The compound of Formula I as the 2-methyl-2-propanamine salt is found to have % regression values as provided in Table 3.

TABLE 3
Evaluation of the Compound of Formula I as the 2-Methyl-
2-Propanamine Salt in Neuroblastoma Xenograft Models

| | | | % Regression (−) | | |
|---|---|---|---|---|---|
| | Xenograft | | at End of | | % Bodyweight |
| Model | Type | N | Treatment | p-value | Change |
| KELLY | CDX* | 5 | −59.4 | <0.001 | −5.3 |
| SH-SY5Y | CDX | 5 | −78.8 | <0.001 | −1.1 |
| IMR-32 | CDX | 4 | −94.3 | <0.001 | 4.7 |

CDX refers to Cell Derived Xenograft Type.
N refers to # of replicates.

These results indicate that the compound of Formula I as the 2-methyl-2-propanamine salt demonstrates significant anti-tumor activity in human NBL xenograft models. The compound of Formula I as the 2-methyl-2-propanamine salt is effective as a single agent in 100% (33) of the pediatric NBL in vivo mouse models tested, with results ranging from stable disease to complete response.

1. A method of treating neuroblastoma in a patient comprising administering to a patient in need of such treatment an effective amount of a compound which is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid or a pharmaceutically acceptable salt thereof.

2. The method according to claim 1 wherein the compound is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid.

3. The method according to claim 1 wherein the compound is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt.

4. The method according to claim 1 wherein the compound is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt.

5. A method of treating neuroblastoma in a patient comprising administering to a patient in need thereof an effective amount of a pharmaceutical composition comprising a compound which is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, or a pharmaceutically acceptable salt thereof, and a pharmaceutically acceptable carrier, diluent or excipient.

6. The method of claim 5 wherein the composition comprises a compound which is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid.

7. The method of claim 5 wherein the composition comprises a compound which is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt.

8. The method of claim 5 wherein the composition comprises a compound which is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt.

9. A compound which is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, or a pharmaceutically acceptable salt thereof, for use in the treatment of neuroblastoma.

10. The compound for use according to claim 9 which is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid.

11. The compound for use according to claim 9 which is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:2-methylpropan-2-amine (1:1) salt.

12. The compound according to claim 9 which is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid:amine (1:1) salt.

13. Use of a compound, which is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid, or a pharmaceutically acceptable salt thereof, for manufacture of a medicament for the treatment of neuroblastoma.

14. The use according to claim 13 wherein the compound is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid.

15. The use according to claim 13 wherein the compound is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid 2-methylpropan-2-amine (1:1) salt.

16. The use according to claim 13 wherein the compound is (2R,4R)-1-[(3-chloro-2-fluoro-phenyl)methyl]-4-[[3-fluoro-6-[(5-methyl-1H-pyrazol-3-yl)amino]-2-pyridyl]methyl]-2-methyl-piperidine-4-carboxylic acid amine (1:1) salt.
